(19) United States
(12) Patent Application Publication
(10) Pub. No.: US 2008/0051628 A1
(43) Pub. Date: Feb. 28, 2008

(54) Handheld portable medical viewing assembly for displaying medical images during medical procedures and intubation stylet

(76) Inventors: Eugeny Pecherer, Netanya (IL); Shiri Soffer, Tel Aviv (IL)

Correspondence Address: Harold L. Novick;NATH & ASSOCIATES PLLC, 112 S. West Street, Alexandria, VA, 22312, US

(21) Appl. No.: 11/898,114

(22) Filed: Sep. 10, 2007

Related U.S. Application Data
(63) Continuation-in-part of application No. PCT/IL2006/000290, filed on Mar. 2, 2006.

(30) Foreign Application Priority Data
Mar. 8, 2005 (IL) 167,302
Mar. 8, 2005 (IL) 167,303
Dec. 14, 2005 (IL) 172,569

(51) Int. Cl. A61B 1/04

(52) U.S. Cl. 600/112

(57) ABSTRACT

Adapter for mounting a handheld portable consumer device having a built-in digital camera and a built-in display screen on a handheld portable endoscope for rendering a handheld portable medical viewing assembly for displaying medical images during a medical procedure, for recording same for subsequent processing, and the like. An intubation stylet preferably entirely made from shape memory metal and fashioned into a J-shaped elongated member having a pre-bent arcuate leading portion with a rectangular cross section designed to facilitate a successful first attempt intubation procedure.

### CROSS REFERENCE TO RELATED APPLICATIONS

This application is a continuation in part application of a PCT Application No. PCT/IL2006/000290 filed Mar. 2, 2006 which designated the United States, the entire contents of which are incorporated herein by reference.

### FIELD OF THE INVENTION

The invention pertains to handheld portable medical viewing assemblies for displaying medical images during medical procedures and intubation stylets.

### BACKGROUND OF THE INVENTION

Handheld portable laryngoscopes with an optical system for viewing a subject's laryngeal region during endotracheal intubation are illustrated and described in inter alia U.S. Pat. No. 4,086,919 to Bullard, U.S. Pat. No. 4,306,547 to Lowell, U.S. Pat. No. 4,901,708 to Lee, U.S. Pat. No. 5,263,472 to Ough, and U.S. Pat. No. 5,873,818 to Rothfels. Handheld portable video laryngoscopes for displaying medical images during endotracheal intubation are illustrated and described in inter alia U.S. Pat. No. 5,827,178 to Berall, U.S. Pat. No. 6,652,453 to Smith et al., U.S. Pat. No. 6,840,903 to Mazzei et al. (see FIG. 5), U.S. Pat. No. 6,929,000 to Hill, and US Patent Application Publication No. 2003/0195390 to Graumann. Medical viewing assemblies for remote displaying of medical images during endotracheal intubation are illustrated and described inter alia in U.S. Pat. No. 6,123,666 to Wrenn et al., U.S. Pat. No. 6,354,993 to Kaplan et al., and U.S. Pat. No. 6,653,447 to Pacey. Such medical viewing assemblies are commercially available from KARL STORZ GmbH & Co. KG, Tuttlingen, Germany, (www.karlstorz.com), and GlideScope® Video Intubations System commercially available from Saturn Biomedical Systems Inc., Burnaby B. C., Canada (www.saturnbiomedical.com). Said twelve U.S. patents and one U.S. patent application publication are incorporated herein in their entireties by reference.

Intubation stylets for assisting in endotracheal intubations are intended to be inserted into endotracheal tubes with Internal Diameters (IDs) ranging from about 4 mm to about 6 mm for pediatric sized tubes and 7 mm to about 8.5 mm for adult sized tubes. Conventional intubation stylets are fashioned as malleable elongated members of uniform circular cross section along their entire lengths and are intended to be manually bent to a desired shape prior to an endotracheal intubation. Intubation stylets for insertion in pediatric sized intubation tubes typically have a diameter of about 3 mm whilst intubation stylets for insertion in adult sized intubation tubes typically have a diameter of about 4.5 mm which leads to undesirable free play for a successful first attempt endotracheal intubation. Exemplary intubation stylets are illustrated and described in U.S. Pat. No. 3,996,939 and U.S. Pat. No. 5,095,888 whilst U.S. Pat. No. 5,259,377 illustrates and describes an endotracheal tube stylet enabling a user to selectively deflect or induce curvature to an elongated member during an intubation procedure, said three U.S. patents being incorporated herein in their entireties by reference.

### SUMMARY OF THE INVENTION

The first aspect of the present invention is directed toward adapters for removably mounting a handheld portable consumer device including a built-in digital camera and a built-in display screen on an endoscope including an optical system for converting same to a handheld portable medical viewing assembly for displaying medical images during medical procedures, recording medical images for processing purposes, and the like. The present invention can be implemented using a wide range of suitable handheld portable consumer devices including inter alia standalone digital cameras, electronic magnifying devices, PDAs, mobile telephones, and the like. Medical viewing assemblies in accordance with the present invention can have comparable functionality as dedicated medical viewing assemblies but at a greatly reduced cost. The present invention is readily applicable to a wide range of endoscopes including inter alia laryngoscopes, bronchoscopes, proctoscopes, colonoscopes, and the like.

The second aspect of the present invention is directed toward an intubation stylet preferably entirely made from shape memory metal and fashioned into a J-shaped elongated member having a pre-bent arcuate leading portion with a rectangular transverse cross section to facilitate a successful first attempt endotracheal intubation. The rectangular transverse cross section also facilitates the use of the same intubation stylet for different diameters of endotracheal tubes. The intubation stylets can emit illumination light at their distal ends and/or optionally be provided with a tube for delivering oxygen or suction purposes. The illumination light can be provided by either a light transmitting fiber optic cable, or an electrical light source, for example, an LED.

### BRIEF DESCRIPTION OF THE DRAWINGS

In order to understand the invention and to see how it can be carried out in practice, preferred embodiments will now be described, by way of non-limiting examples only, with reference to the accompanying drawings, in which similar parts are likewise numbered, and in which:

FIG. 1 is a front perspective view of a first preferred embodiment of a medical viewing assembly in its assembly/disassembly position, the medical viewing assembly including an adapter for removably mounting a digital camera with a telescopic objective lens on a laryngoscope for displaying medical images of a subject's laryngeal region during endotracheal intubation;

FIG. 2 is a rear perspective view of FIG. 1's medical viewing assembly;

FIG. 3 is an exploded view of FIG. 1's medical viewing assembly;

FIG. 4 is a front view of the adapter's body member;

FIG. 5 is a front view of the laryngoscope's eyepiece;

FIG. 6 is a longitudinal cross section of FIG. 1's medical viewing assembly of along line A-A in FIG. 1 with a retracted objective lens;

FIG. 7 is a longitudinal cross section of FIG. 1's medical viewing assembly along line A-A in FIG. 1 with a fully protruding objective lens;

FIG. 8 is a front perspective view of a second preferred embodiment of a medical viewing assembly in its assembly/disassembly position, the medical viewing assembly including an adapter for removably mounting a digital camera with a non-telescopic zoom lens on a laryngoscope for displaying medical images of a subject's laryngeal region during endotracheal intubation;

FIG. 9 is a front perspective view of a third preferred embodiment of a medical viewing assembly including an adapter for removably mounting a digital camera on a laryngoscope for displaying medical images of a subject's laryngeal region during endotracheal intubation;

FIG. 10 is a perspective view of a preferred embodiment of an intubation stylet;

FIGS. 11 and 12 are transverse cross sections of FIG. 10's intubation stylet along lines B-B and C-C in FIG. 10;

FIG. 13 is a perspective view of an intubation stylet with a fiber optic cable for illuminating a subject's laryngeal region during endotracheal intubation;

FIG. 14 is a perspective view of an intubation stylet with an electrical powered light source for illuminating a subject's laryngeal region during endotracheal intubation; and

FIG. 15 is a perspective view of an intubation stylet for delivering oxygen to a subject.

### DETAILED DESCRIPTION OF PREFERRED EMBODIMENTS OF THE PRESENT INVENTION

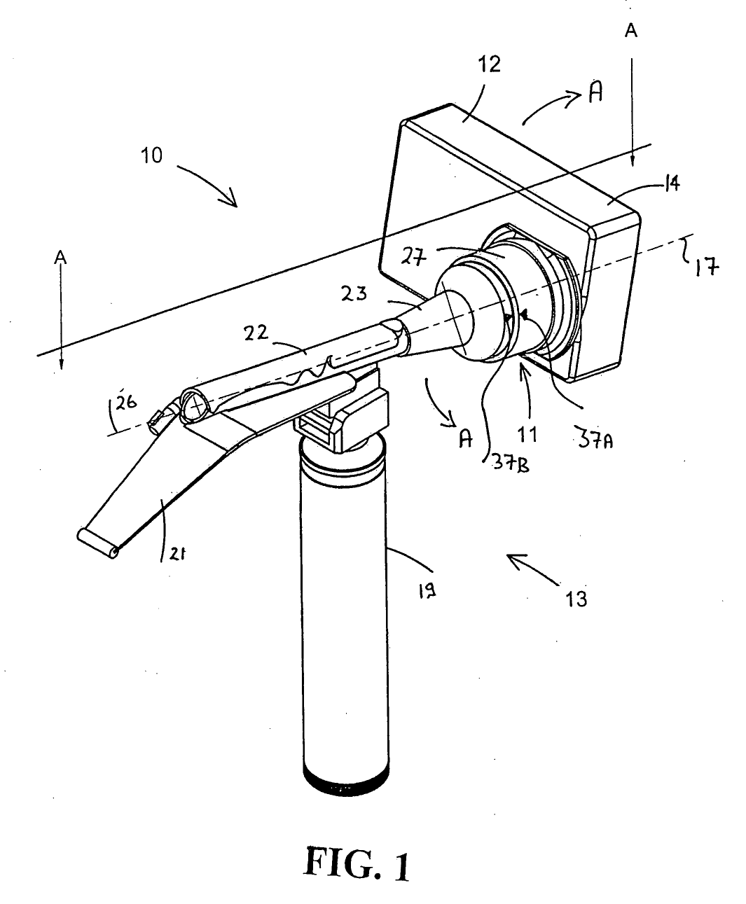

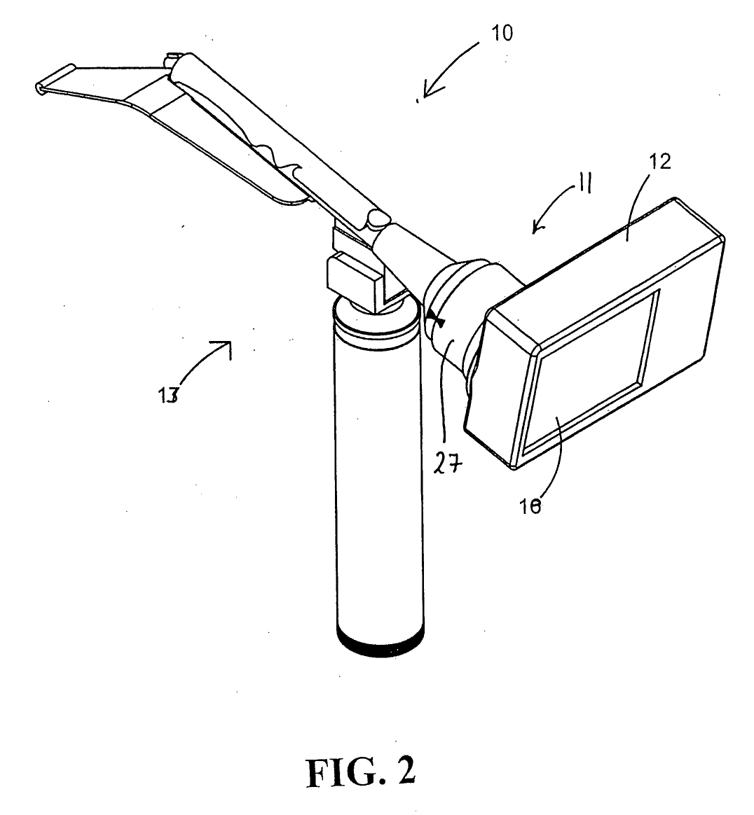

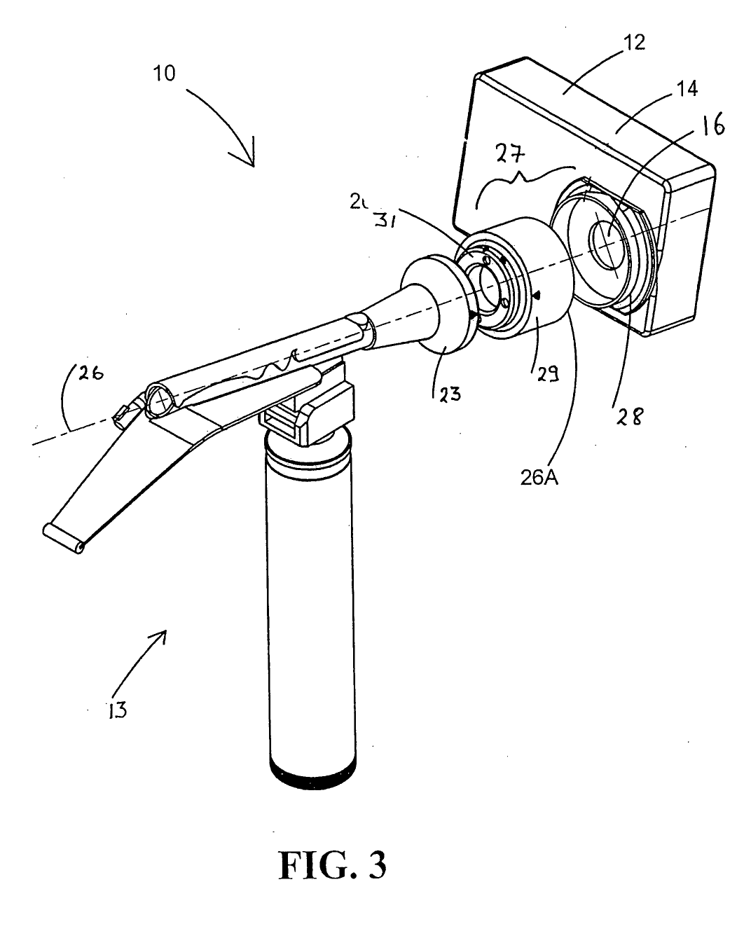

FIGS. 1 to 9 illustrate the use of adapters for converting handheld portable endoscopes to handheld portable medical viewing assemblies for displaying medical images during medical procedures, recording medical images for processing purposes, and the like. Exemplary handheld portable endoscopes include inter alia laryngoscopes, bronchoscopes, proctoscopes, colonoscopes, and the like. The adapters are intended to be used with a wide range of suitable handheld portable consumer devices having a built-in digital camera and a built-in display screen including inter alia standalone digital cameras, electronic magnifying devices, PDAs, mobile telephones, and the like.

The adapters preferably include a body member for permanent attachment to a handheld portable consumer device to encircle its objective lens and a purpose built eyepiece. The body member and the eyepiece preferably include a mechanical coupling arrangement enabling manual rotation of the consumer device relative to the endoscope and having a single assembly position for enabling mounting of the consumer device on the endoscope and dismounting therefrom. Also, the base member and the eyepiece include a visible indication arrangement to assist a user to rotationally align the consumer device with the endoscope to the single assembly position. Eyepieces can be permanently attached on suitable endoscopic optical systems, for example, the TRU-VIEW™ EVO optical view tube commercially available from the Assignees of the present invention. Alternatively, eyepieces can be replaceable components with a distal end for screw threading onto a corresponding endoscopic eyepiece adapter. The latter arrangement enables conversion of existing endoscopes to handheld portable medical viewing assemblies in accordance with the present invention.

The mechanical coupling arrangement can be supplemented by a magnetic coupling arrangement for intimately juxtaposing the base member and the endoscope's eyepiece in the desired optical alignment. The magnetic coupling arrangement is preferably implemented by a base member including at least one magnet and an endoscope's eyepiece including a magnetic stainless steel ring.

The digital cameras can include an objective lens with a fixed focal length or a telescopic objective lens for optical zooming. Adapters are preferably shaped and dimensioned to enable full zooming capabilities of telescopic objective lens, namely, to space an eyepiece away from a digital camera's front surface to preclude an eyepiece impeding optical zooming. In the case of enabling optical zooming, body members preferably include a base member for permanent attachment on a consumer device and an extension member removably mountable on the base member. This arrangement reduces the weight of the adapter which is permanently added to the consumer device and also facilitates cleaning its objective lens.

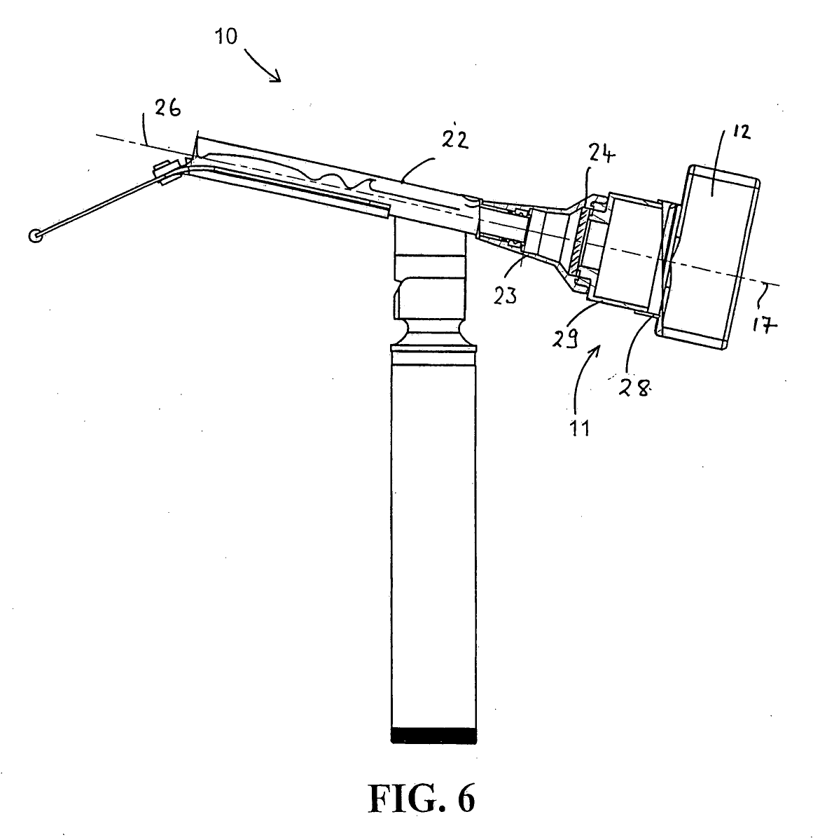

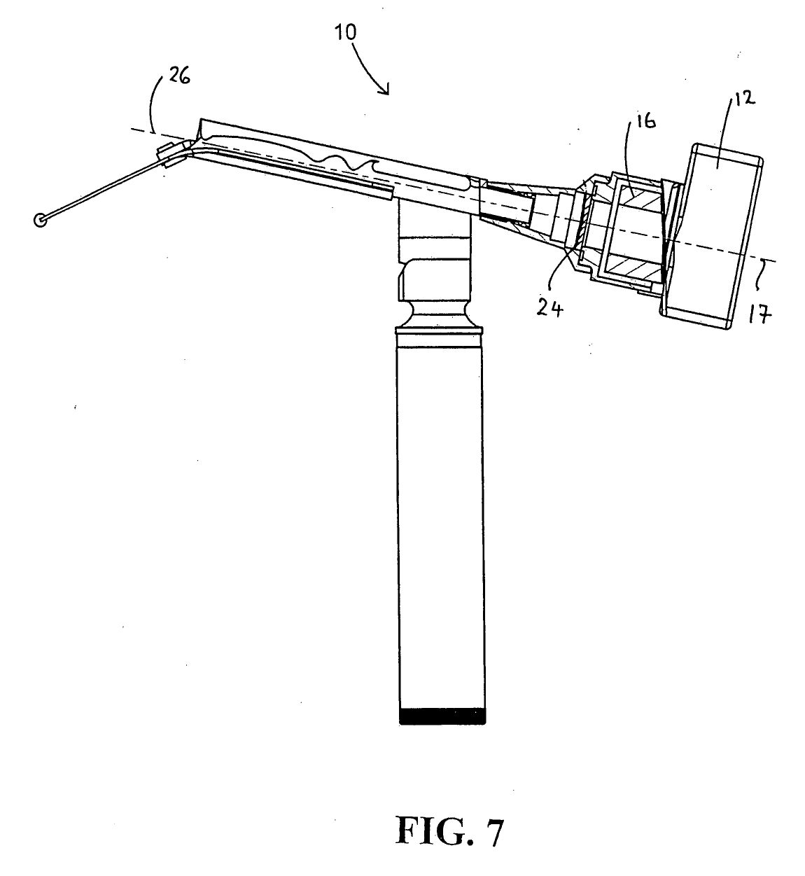

FIGS. 1-7 show a handheld portable medical viewing assembly 10 including an adapter 11 for removably mounting a handheld portable digital camera 12 on a handheld portable laryngoscope 13 for displaying medical images of a subject's laryngeal region during endotracheal intubation. The digital camera 12 includes a housing 14 having a telescopic objective lens 16 with an optical axis 17 and a display screen 18. An exemplary digital camera is the Premier Digital Camera DS-5341 having a telescopic objective lens retracted inside its housing when powered down (see FIG. 6), and having a maximum outward protrusion of about 25 millimeters (see FIG. 7). The laryngoscope 13 includes a handle 19, a blade 21, and an optical view tube 22 with a permanently attached eyepiece 23 including an eyepiece lens 24 and an optical axis 26. The eyepiece 23 is typically formed from non-magnetic material, for example, aluminum, plastic, and the like. Exemplary laryngoscopes include TRUVIEW™ laryngoscopes commercially available from the Applicants of the present application Truphatek International Ltd, Netanya, Israel. Online details are available at http://www.truphatek.com/default.php?p=products&p1=2&p2=88.

The adapter 11 includes a body member 27 for co-axial alignment of the digital camera's objective lens 16 with the optical view tube 22 in the assembled state of the medical viewing assembly 10. The body member 27 preferably includes a base member 28 for preferably permanently mounting on the housing 14 to encircle its objective lens 16 and a tubular extension member 29 for screw thread attachment on the base member 28. The base member 28 can be glued on the housing 14, screwed on, and the like.

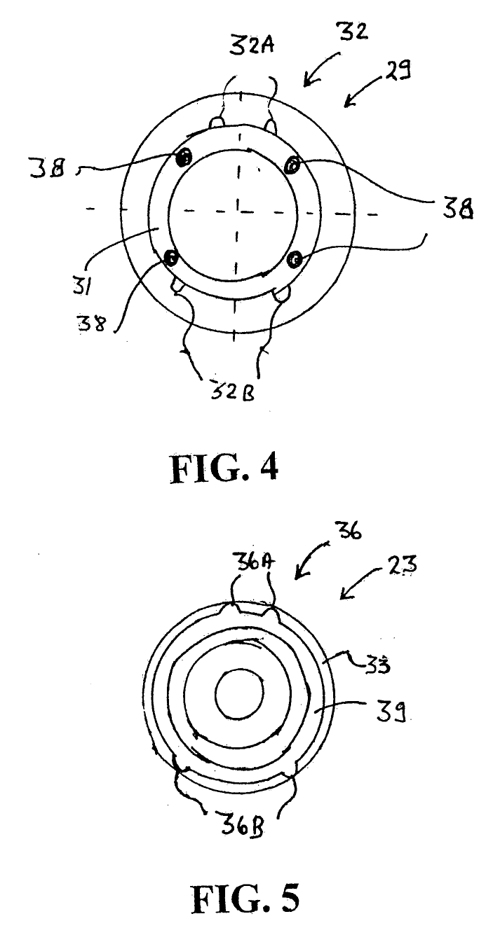

The extension member 29 includes a leading ring 31 for insertion into the eyepiece 23. The leading ring 31 includes a pin arrangement 32 of one pair of radial outward pins 32A having a greater separation than the other pair 32B. The eyepiece 23 includes a proximal peripheral flange 33 facing the body member 27 on assembly of the medical viewing assembly 10 (see FIG. 4). The peripheral flange 33 includes a cutout arrangement 36 including pairs of cutouts 36A and 36B corresponding to the pin arrangement 32 (see FIG. 5) for affording a single assembly position for mounting the digital camera 12 on the laryngoscope 13 on axial insertion of the leading ring 31 into the eyepiece 23. The body member 27 and the eyepiece 23 provide mechanical coupling between the digital camera 12 and the laryngoscope 13 for enabling manual rotation through a near full circle as denoted by arrows A in FIG. 1 thereby affording convenient displaying of medical images.

The body member 27 has a visual marking 37A and the eyepiece 23 has a corresponding visual marking 37B thereby enabling a user to readily align the body member 27 with the eyepiece 23 to enable mounting of digital camera 12 on the laryngoscope 13 and dismounting therefrom. The leading ring 31 is provided with four magnets 38 (see FIG. 4) and the eyepiece 23 is provided with a magnetic stainless ring 39 (see FIG. 5) for affording a magnetic coupling arrangement for magnetically coupling the digital camera 12 to the laryngoscope 13 in the desired optical alignment.

The use of the medical viewing assembly 10 is as follows:

A user attaches the base member 28 onto the housing 14 to encircle its objective lens 16. The user mounts the extension member 29 on the base member 28 to assemble the body member 27. The user aligns the laryngoscope's eyepiece's cutout arrangement 36 with the body member's pin arrangement 32 to enable insertion of the body member's leading ring 31 into the laryngoscope's eyepiece 23 in its single assembly position. The user rotates the digital camera 12 with respect to the laryngoscope 13 to a preferred viewing position thereby securing the digital camera 12 on the laryngoscope 13. The user can view medical images on the display screen 18 during an endotracheal intubation. The user can use the digital camera 12 for taking still and/or video images, and the like.

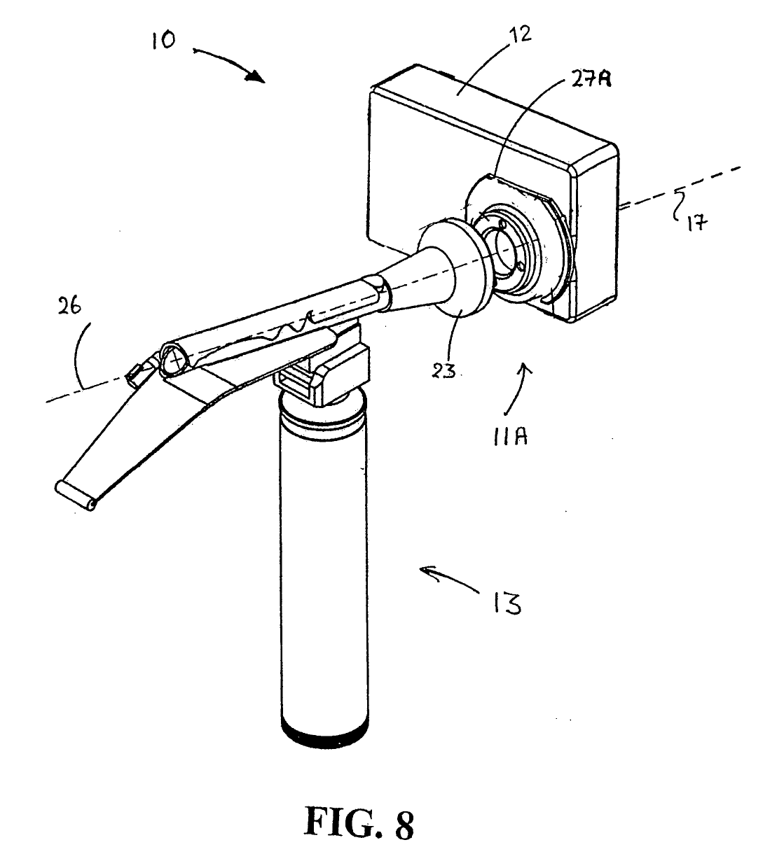

FIG. 8 shows a handheld portable medical viewing assembly 10 including an adapter 11A similar to the adapter 11 but having a one piece body member 27A of shorter axial length suitable for use with digital cameras having a non-telescopic objective lens.

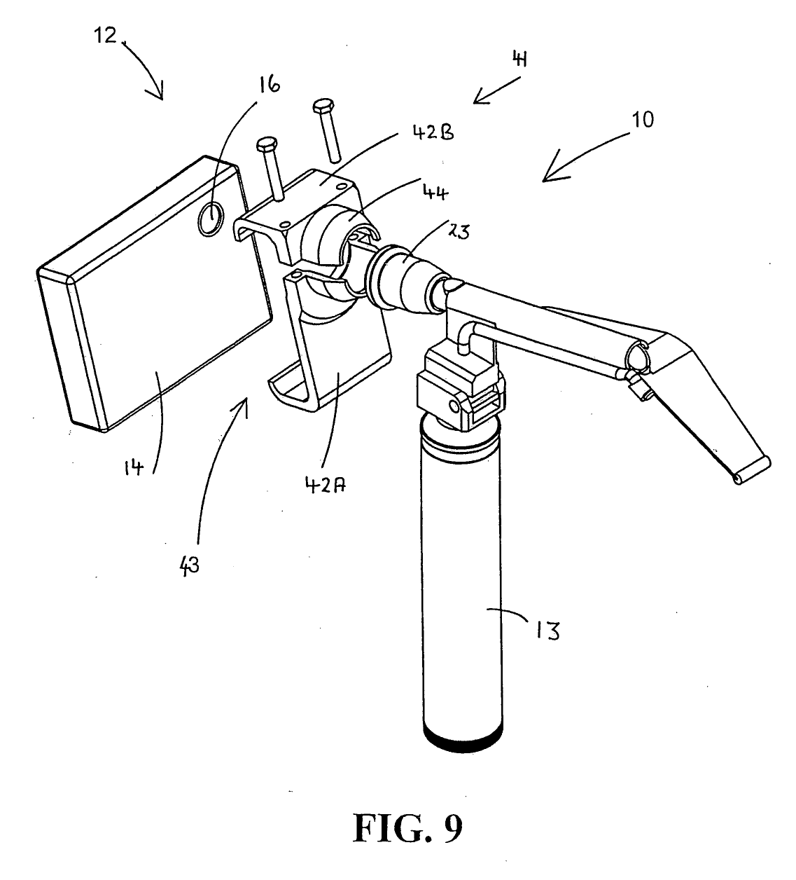

FIG. 9 shows a handheld portable medical viewing assembly 10 including an adapter 41 similar to the adapter 11 but formed from a pair of L-shaped flange members 42A and 42B for defining a U-shaped channel 43 for secure mounting on a digital camera's housing 13 and a tubular aperture 44 for secure mounting on an eyepiece 23 on screw clamping the L-shaped flange members 42A and 42B together. The adapter 41 enables clockwise and counter clockwise rotation of the digital camera 12 with respect to the laryngoscope 13 to a preferred viewing position.

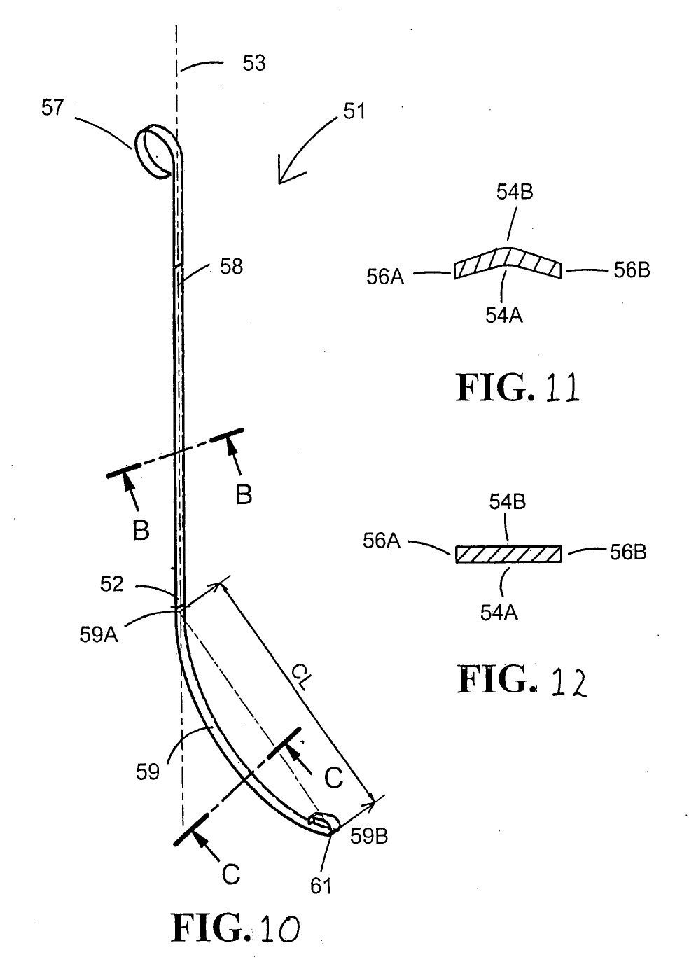

FIGS. 10-12 shows an intubation stylet 51 made from a rolled metal strip of shape memory material, for example, spring steel, and the like, having a rectangular cross section. The intubation stylet 51 has a generally J-shaped elongated member 52 with a longitudinal axis 53, and a rectangular transverse cross section perpendicular to the longitudinal axis 53 with major front and rear surfaces 54A and 54B and minor side surfaces 56A and 56B (see FIG. 11). The rectangular transverse cross section affords flexibility in the Y-Z plane and rigidity in the X-Z plane for facilitating successful first attempt intubations. The elongated member 52 includes a hand held trailing portion 57, a generally straight intermediate portion 58, and a pre-bent arcuate leading portion 59 with a leading tip 61 curled back upon itself. The leading portion 59 has opposite ends 59A and 59B defining an arc length in the order of about 15 cm and a chord length CL of about 13 cm for an adult sized intubation stylet 51. The intermediate portion 58 preferably is formed with a V-shaped cross section in the X-Y plane to afford greater rigidity in the Y-Z plane (see FIG. 12). The generally straight intermediate portion 58 may be formed with a circular cross section in the X-Y plane. Alternatively, only the pre-bent arcuate leading portion 59 may be formed from shape memory material and suitably attached to an intermediate portion 58 made from non-shape memory material by soldering, and the like.

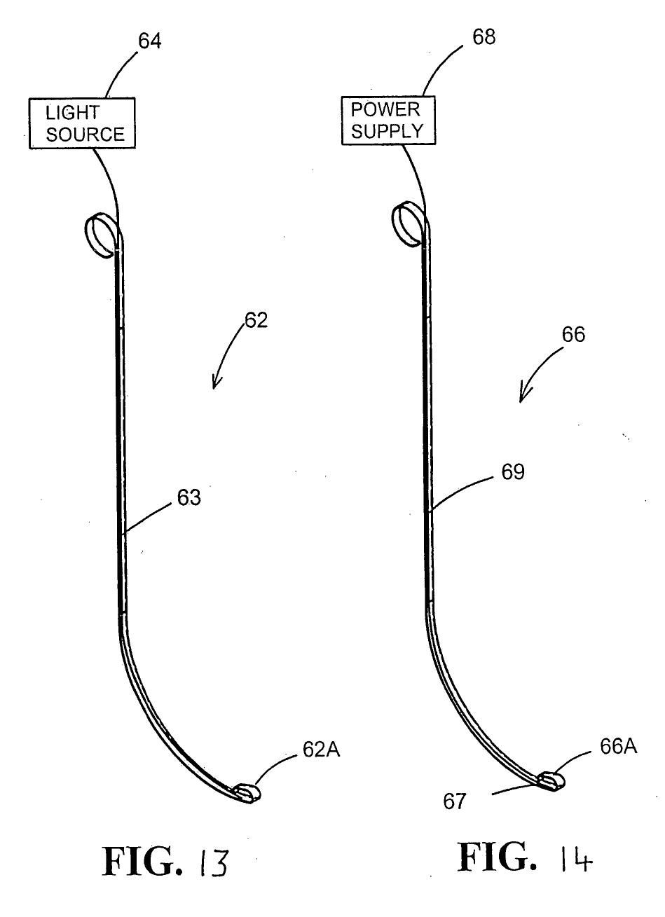

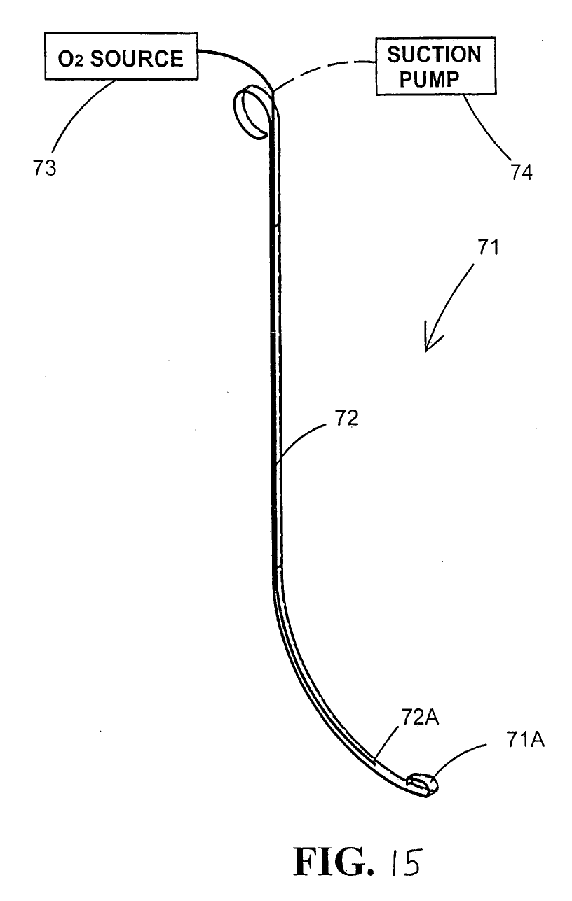

FIG. 13 shows an intubation stylet 62 similar to the intubation stylet 51 and additionally having a fiber optic cable 63 connected to a light source 64 and terminating at its leading tip 62A for illuminating a subject's laryngeal region during endotracheal intubation. FIG. 14 shows an intubation stylet 66 similar to the intubation stylet 51 and having an electrical powered light source 67 at its leading tip 66A connected to a power supply 68 via an electrical wire 69 for illuminating a subject's laryngeal region during endotracheal intubation. FIG. 15 shows an intubation stylet 71 similar to the intubation stylet 51 and additionally having a tube 72 having an opening 72A at the stylet's distal end 71A in flow communication with either an oxygen source 73 for delivering oxygen to a subject during an endotracheal intubation or a suction pump 74 for removing undesirable liquids from a subject's tracheal region during endotracheal intubation.

While the invention has been described with respect to a limited number of embodiments, it will be appreciated that many variations, modifications, and other applications of the invention can be made within the scope of the appended claims.

1. Adapter for use with (a) a handheld portable consumer device including a housing having a built-in digital camera with an objective lens and an optical axis, and a built-in display screen for displaying visual images, and (b) a handheld portable endoscope including an eyepiece and an optical system with an optical axis for viewing a medical image,
the adapter comprising a body member for removably mounting the consumer device on the endoscope with the digital camera's optical axis in co-axial alignment with the endoscope's optical axis for rendering a handheld portable medical viewing assembly for displaying medical images during a medical procedure,
the consumer device being manually rotatable with respect to the endoscope in an assembled state of the handheld portable medical viewing assembly during a medical procedure to a preferred viewing position.

2. The adapter according to claim 1 wherein said body member and the endoscope's eyepiece include a mechanical coupling arrangement enabling said manual rotation of the consumer device relative to the endoscope and having at least one assembly position for enabling axial mounting of the consumer device on the endoscope and axial dismounting therefrom, and said body member and the endoscope's eyepiece each include a visible indication to assist a user to rotationally align said body member with the endoscope's eyepiece to enable said mounting of the consumer device on the endoscope and dismounting therefrom.

3. The adapter according to claim 2 wherein said body member includes a leading ring with a pin arrangement of at least one outward radial pin and the endoscope's eyepiece includes a proximal peripheral flange facing said body member on assembly of the medical viewing assembly and said proximal peripheral flange includes a cutout arrangement corresponding to said pin arrangement such that rotational alignment between said pin arrangement and said cutout arrangement enables axial insertion of said leading ring into the endoscope's eyepiece at said at least one assembly position.

4. The adapter according to claim 3 wherein said pin arrangement and said cutout arrangement provide a single assembly position for enabling axial mounting of the consumer device on the endoscope and axial dismounting therefrom.

5. The adapter according to claim 1 wherein said body member and the endoscope's eyepiece include a magnetic coupling arrangement for magnetically coupling the consumer device to the endoscope in the desired optical alignment.

6. The adapter according to claim 1 wherein the digital camera has a telescopic objective lens and said body member is dimensioned to enable full optical zoom of the digital camera's telescopic objective lens in the assembled state of the medical viewing assembly.

7. The adapter according to claim 1 wherein said body member is intended for permanent attachment onto the housing for encircling the housing's objective lens.

8. The adapter according to claim 6 wherein said body member includes a base member for permanent attachment on the housing and a tubular extension member for removable attachment on said base member and
said extension member and the endoscope's eyepiece include a mechanical coupling arrangement enabling said manual rotation of the consumer device relative to the endoscope and having at least one assembly position for enabling mounting of the consumer device on the endoscope and dismounting therefrom and said extension member and the endoscope's eyepiece each include a visible indication to assist a user to align said extension member with the endoscope's eyepiece to enable said mounting of the consumer device on the endoscope and dismounting therefrom.

9. The adapter according to claim 8 wherein said extension member includes a leading ring with a pin arrangement of at least one outward radial pin and the endoscope's eyepiece includes a proximal peripheral flange facing said extension member on assembly of the medical viewing assembly and said proximal peripheral flange includes a cutout arrangement corresponding to said pin arrangement such that rotational alignment between said pin arrangement and said cutout arrangement enables axial insertion of said leading ring into the endoscope's eyepiece at said at least one assembly position.

10. The adapter according to claim 9 wherein said pin arrangement and said cutout arrangement provide a single assembly position for enabling axial mounting of the consumer device on the endoscope and axial dismounting therefrom.

11. The adapter according to claim 8 wherein said extension member and the endoscope's eyepiece include a magnetic coupling arrangement for magnetically coupling the consumer device to the endoscope in the desired optical alignment.

12. The adapter according to claim 1 wherein said body member includes a pair of L-shaped flange members for defining a U-shaped channel for secure mounting on the housing and a tubular aperture for secure mounting on the endoscope's eyepiece on clamping said L-shaped flange members together.

13. A handheld portable medical viewing assembly for displaying medical images during a medical procedure, the assembly comprising:
(a) a handheld portable consumer device including a housing having a built-in digital camera with an objective lens and an optical axis, and a built-in display screen for displaying visual images;
(b) a handheld portable endoscope including an optical system having an eyepiece and an optical axis for viewing a medical image; and
(c) an adapter for removably mounting said consumer device on said endoscope with said digital camera's optical axis in co-axial alignment with said endoscope's optical axis and enabling manual rotation of said consumer device with respect to said endoscope in an assembled state of the handheld portable medical viewing assembly during a medical procedure to a preferred viewing position.

14. The assembly according to claim 13 wherein said adapter and the endoscope's eyepiece include a mechanical coupling arrangement enabling said manual rotation of said consumer device relative to said endoscope and having at least one assembly position for enabling mounting of said consumer device on said endoscope and dismounting therefrom and
said adapter and said endoscope's eyepiece each include a visible indication to assist a user to rotationally align said adapter with said endoscope's eyepiece to enable said mounting of said consumer device on said endoscope and dismounting therefrom.

15. The assembly according to claim 13 wherein said adapter includes a pin arrangement with at least one outward radial pin and said endoscope's eyepiece includes a proximal peripheral flange facing said adapter on assembly of the medical viewing assembly and said proximal peripheral flange includes a cutout arrangement corresponding to said pin arrangement such that rotational alignment between said pin arrangement and said cutout arrangement enables axial insertion of said leading ring into the endoscope's eyepiece at said at least one assembly position.

16. The assembly according to claim 15 wherein said pin arrangement and said cutout arrangement provide a single assembly position for enabling axial mounting of said consumer device on said endoscope and axial dismounting therefrom.

17. The assembly according to claim 13 wherein said adapter and said endoscope's eyepiece include a magnetic coupling arrangement for magnetically coupling said consumer device to said endoscope in the desired optical alignment.

18. The assembly according to claim 13 wherein said digital camera has a telescopic objective lens and said adapter is dimensioned to enable full optical zoom of said digital camera's telescopic objective lens in the assembled state of the medical viewing assembly.

19. The assembly according to claim 13 wherein said adapter includes a body member for permanent attachment onto said consumer device for encircling said digital camera's objective lens.

20. The assembly according to claim 13 wherein said adapter includes a base member for permanent attachment onto said consumer device for encircling said digital camera's objective lens and a tubular extension member for removable attachment on said base member and
said extension member and said endoscope's eyepiece include a mechanical coupling arrangement enabling said manual rotation of said consumer device relative to said endoscope and having at least one assembly position for enabling mounting of said consumer device on said endoscope and dismounting therefrom and said extension member and said endoscope's eyepiece each include a visible indication to assist a user to align said extension member with said endoscope's eyepiece to enable said mounting of said consumer device on said endoscope and dismounting therefrom.

21. The assembly according to claim 20 wherein said extension member includes a pin arrangement with at least one outward radial pin and said endoscope's eyepiece includes a proximal peripheral flange facing said adapter on assembly of the medical viewing assembly and said proximal peripheral flange includes a cutout arrangement corresponding to said pin arrangement such that rotational alignment between said pin arrangement and said cutout arrangement enables axial insertion of said leading ring into the endoscope's eyepiece at said at least one assembly position.

22. The assembly according to claim 20 wherein said extension member and the endoscope's eyepiece include a magnetic coupling arrangement for magnetically coupling the consumer device to the endoscope in the desired optical alignment.

23. The assembly according to claim 13 wherein said adapter includes a pair of L-shaped flange members for defining a U-shaped channel for secure mounting on said housing and a tubular aperture for secure mounting on said endoscope's eyepiece on clamping said L-shaped flange members together.

24. The assembly according to claim 13 wherein the eyepiece is a replaceable component including a distal end for screw threading into endoscopic equipment.

25. The assembly according to claim 13 wherein the endoscope includes a permanently attached eyepiece.

26. The assembly according to claim 25 wherein the endoscope is constituted by a laryngoscope including an optical view tube with a permanently connected eyepiece.

27. An intubation stylet comprising a generally J-shaped elongated member having a longitudinal axis, and a hand held trailing portion, a generally straight intermediate portion, and a pre-bent arcuate leading portion, at least said leading portion being formed from shape memory material and having a generally rectangular transverse cross section perpendicular to said longitudinal axis.

28. The stylet according to claim 27 wherein said intermediate portion has a V-shaped transverse cross section perpendicular to said longitudinal axis.

29. The stylet according to claim 27 wherein said leading portion terminates in a tip curled back upon itself.

30. The stylet according to claim 27 wherein said elongated member is formed from a rolled metal strip of shape memory material having an initial rectangular cross section along its entire length.

31. The stylet according to claim 27 wherein an adult sized stylet has a leading portion whose opposite ends define an arc length in the order of about 15 cm and a chord length of about 12 cm.

32. The stylet according to claim 27, said elongated member having a distal end, said stylet further including an emitting illumination light at said distal end of said elongated member for illuminating a subject's laryngeal region during endotracheal intubation.

33. The stylet according to claim 27 and further comprising a tube with an opening at its distal end.
